(19) United States
(12) Patent Application Publication
(10) Pub. No.: US 2019/0162589 A1
(43) Pub. Date: May 30, 2019

(54) PHOTOACOUSTIC PROBE AND ATTACHMENT

(71) Applicant: CANON KABUSHIKI KAISHA, Tokyo (JP)

(72) Inventors: Takaaki Nakabayashi, Kawasaki-shi (JP); Toshinobu Tokita, Yokohama-shi (JP)

(21) Appl. No.: 16/199,405

(22) Filed: Nov. 26, 2018

(30) Foreign Application Priority Data
Nov. 29, 2017 (JP) 2017-229009

(51) Int. Cl. G01H 9/00; A61B 5/00; A61B 8/00

(52) U.S. Cl. CPC G01H 9/008; A61B 8/4444; A61B 5/0095

(57) ABSTRACT

A photoacoustic probe includes: a light source which generates light to be irradiated on an object; a probe body including a receiving portion which receives a photoacoustic wave generated from the object in response to irradiation of the light emitted from the light source on the object and a contact portion which contacts the object; and an attachment to which the probe body and the light source are attached, wherein the light source is attachable to and detachable from the attachment from a side different from the object side where the contact portion contacts the object.

### BACKGROUND OF THE INVENTION

### Field of the Invention

The present invention relates to a photoacoustic probe and an attachment.

### Description of the Related Art

In the medical field, researches have been conducted on photoacoustic devices for imaging the internal forms and functions of an object using the photoacoustic effect. In a conventional photoacoustic device, a controller in the photoacoustic device transmits a signal to a driving circuit for a light source in response to an instruction from a user, and a pulsed beam is emitted from the source. The pulsed beam is irradiated on the object through light guiding means, and then a photoacoustic wave is generated. The photoacoustic wave is received by a receiving element included in a probe in contact with the object and converted into an electrical signal called a photoacoustic signal. The controller carries out signal processing and image reconstruction processing to the photoacoustic signal and calculates the initial sound pressure and the absorption coefficient of the object. Using absorption coefficients derived from pulsed light having different wavelengths from one another, oxygen saturation can be calculated. The photoacoustic device presents the user with a diagnostic image based on these kinds of object information.

In some of the photoacoustic devices, a probe provided with a receiving element may further include an irradiating portion which irradiates the object with light. Such a photoacoustic probe, particularly a hand-held probe is preferably used. When a user such as a medical doctor measures an object using a hand-held photoacoustic probe, the user irradiates light while pressing the receiving portion of the probe against the object and receives a photoacoustic wave.

Japanese Patent Application Publication No. 2016-049215 discloses a photoacoustic probe comprising an LED light source unit which irradiates light and a receiving element which receives a photoacoustic wave. Japanese Patent Application Publication No. 2016-049215 also discloses a photoacoustic probe comprising a light irradiating unit including an LED light source unit in the form of an attachment which can be attached to and detached from an ultrasonic probe body. In this way, the waveform can easily be switched among a plurality of waveforms, which eliminates the necessity of an expensive variable waveform light source, so that the cost can be reduced.

### SUMMARY OF THE INVENTION

Here, in order to obtain the oxygen saturation of an object highly accurately, light with a plurality of waveforms are preferably irradiated on the object in the same site. Therefore, when the waveform is switched, the positional relation between the photoacoustic probe and the object should be as unchanged as possible. To this end, the light irradiating unit should desirably be attached and detached while the photoacoustic probe is in contact with the object in the configuration disclosed in Japanese Patent Application Publication No. 2016-049215.

However, in the photoacoustic probe disclosed in Japanese Patent Application Publication No. 2016-049215, the attachment is configured to slide in the direction perpendicular to the direction in which the ultrasonic probe body transmits/receives an acoustic wave and fixed to the ultrasonic probe body. Therefore, it is difficult to detach/attach the attachment while the receiving portion of the ultrasonic probe body is in contact with the object, and the probe must temporarily be separated from the object. As a result, the reproducibility of the positional relation between the probe and the object between before and after the waveform switching is lowered.

With the problem in view, it is an object of the present invention to provide a photoacoustic probe having a light irradiating unit replaced depending on the wavelength of irradiation light and allow the unit replacement to be carried out while the probe is in contact with an object.

According to an aspect of the present invention, there is provided a photoacoustic probe including: a light source which generates light to be irradiated on an object; a probe body including a receiving portion which receives a photoacoustic wave generated from the object in response to irradiation of the light emitted from the light source on the object and a contact portion which contacts the object; and an attachment to which the probe body and the light source are attached, wherein the light source is attachable to and detachable from the attachment from a side different from the object side where the contact portion contacts the object.

According to another aspect of the present invention, there is provided a photoacoustic probe including: a light source which generates light to be irradiated on an object; a probe body including a receiving portion which receives a photoacoustic wave generated from the object in response to irradiation of the light emitted from the light source on the object and a contact portion which contacts the object; and an attachment to which the probe body and the light source are attached, wherein the light source is attachable to and detachable from the attachment while the contact portion is in contact with the object.

According to another aspect of the present invention, there is provided an attachment to which a light source and a probe body are attached, the light source generating light to be irradiated on an object, the probe body including a receiving portion which receives a photoacoustic wave generated from the object in response to irradiation of the light emitted from the light source on the object and a contact portion which contacts the object, wherein the light source is attachable to and detachable from the attachment a side different from the object side where the contact portion contacts the object.

According to another aspect of the present invention, there is provided an attachment to which a light source and a probe body are attached, the light source generating light to be irradiated on an object, the probe body including a receiving portion which receives a photoacoustic wave generated from the object in response to irradiation of the light emitted from the light source on the object and a contact portion which contacts the object, wherein the light source is attachable to and detachable from the attachment while the contact portion is in contact with the object.

According to the present invention, a photoacoustic probe having a light irradiating unit replaced depending on the wavelength of irradiation light is provided, and the unit replacement can be carried out while the probe is in contact with an object.

Further features of the present invention will become apparent from the following description of exemplary embodiments with reference to the attached drawings.

### BRIEF DESCRIPTION OF THE DRAWINGS

FIGS. 1A and 1B are views of the structure of a photoacoustic device according to a first embodiment of the present invention;

FIGS. 2A and 2B are views of a state in which a light source unit is turned with respect to an attachment;

FIGS. 3A, 3B and 3C are respectively a perspective view, a side view, and a sectional view of a light source container;

FIGS. 4A and 4B are schematic views of a light source in the process of being inserted into the light source container;

FIG. 5 is a sectional view of an exemplary arrangement in which a light source unit is provided on either side of a probe;

FIGS. 6A and 6B are schematic views of an arrangement in which light from a light source is irradiated in a fixed direction;

FIGS. 7A and 7B are schematic views of another arrangement in which light from a light source is irradiated in a fixed direction;

FIGS. 8A and 8B are views of a photoacoustic device according to a second embodiment of the present invention;

FIGS. 9A to 9D are views of a photoacoustic device according to a third embodiment of the present invention; and

FIGS. 10A to 10D are schematic view of a limit switch.

### DESCRIPTION OF THE EMBODIMENTS

Preferred embodiments of the present invention will be described in conjunction with the accompanying drawings. Note however that the sizes, materials, and shapes of the components, their relative positional arrangements, etc. in the following description should be changed as appropriate depending on the configuration of a device to which the invention is applied or various conditions and are not intended to limit the scope of the invention.

The present invention relates to a technique for detecting an acoustic wave propagated from an object and producing and acquiring characteristic information (object information) on the inside of the object. Therefore, the present invention also relates to a photoacoustic device or a controlling method therefor and an object information acquiring device or a controlling method therefor. The present invention also relates to a method for acquiring object information or a signal processing method. The present invention also relates to an image information processing device or an image information processing method for processing image information on the inside of an object. The present invention relates to a program which allows an information processing device including a hardware resource such as a CPU and a memory to execute any of these methods and a non-transitory computer-readable storage medium storing the program.

The object information acquiring device according to the present invention includes a photoacoustic device based on the photoacoustic effect, and the device receives a photoacoustic wave generated in an object in response to irradiation of light (an electromagnetic wave) on the object and acquire characteristic information on the object as image data. In this case, the characteristic information is information on characteristic values corresponding to a plurality of sites in the object produced using a signal derived from the received photoacoustic wave.

The characteristic information acquired at the time represents a distribution of sources of acoustic waves generated by light irradiation, an initial sound pressure distribution in the object or an optical energy absorption density distribution or an absorption coefficient distribution derived from the initial sound pressure distribution, and concentration distributions of substances which constitute tissues. The concentration distributions of substances may include an oxygen saturation distribution, a total hemoglobin concentration distribution, and an oxidized/reduced hemoglobin concentration distribution.

The characteristic information as the object information in the plurality of sites may be acquired as a two-dimensional or three-dimensional characteristic distribution. The characteristic distribution may be produced as image data representing the characteristic information in the object. The image data may be produced as three-dimensional volume data by image reconstruction.

The acoustic wave according to the present invention is typically an ultrasonic wave and includes an elastic wave called a sound wave or an acoustic wave. A signal (such as an electrical signal) converted from an acoustic wave for example using a transducer is also referred to as an acoustic signal or a reception signal. Note however that the term ultrasonic wave or acoustic wave herein is not intended to limit the wavelengths of these elastic waves. An acoustic wave generated by the photoacoustic effect is referred to as a photoacoustic wave or an optical ultrasonic wave. A signal derived from a photoacoustic wave (such as an electrical signal) is also referred to as a photoacoustic signal. An image produced from a photoacoustic signal for example by image reconstruction is referred to as a photoacoustic image.

### First Embodiment

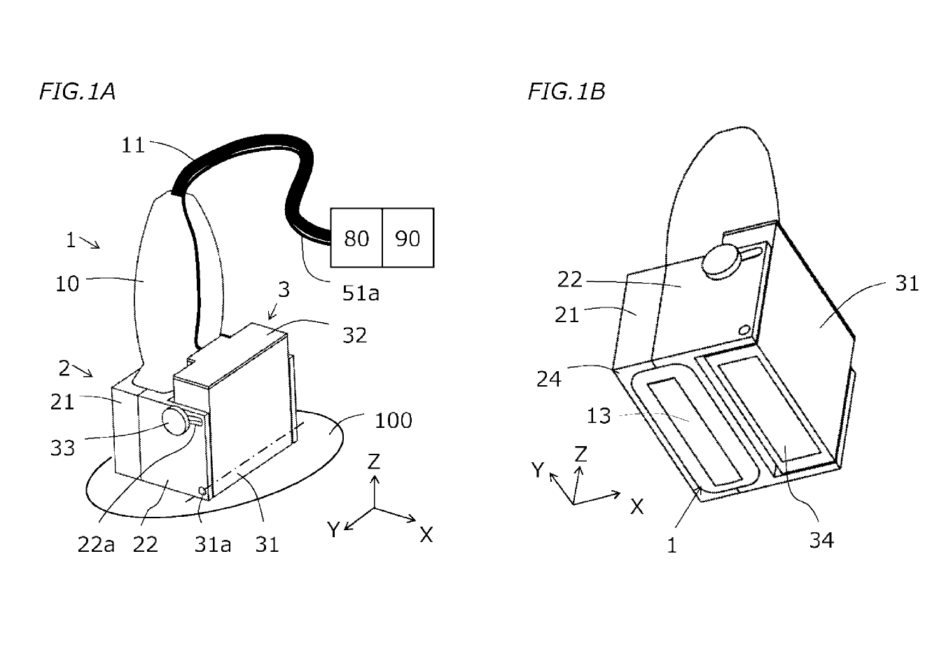

FIGS. 1A and 1B are views of a photoacoustic device according to the embodiment. FIG. 1A is a perspective view from above (from the opposite side to an object as viewed from the side of a photoacoustic probe) showing a state in which the photoacoustic probe of the photoacoustic device is pressed against the object 100. FIG. 1B is a perspective view from below (from the same side as the object as viewed from the side of the photoacoustic probe) of the photoacoustic probe as a part of the photoacoustic device.

The reference numeral 1 designates the ultrasonic probe held by the operator's hand to receive a photoacoustic signal. The probe 1 may be referred to as the “photoacoustic probe” or a state in which the light irradiating unit is mounted to the probe 1 by an attachment may be referred to as a “photoacoustic probe” as will be described. The probe 1 includes a probe body 10 and a probe cable 11 and is connected to an operation unit 80 by a connector which is not shown.

A photoacoustic wave from an object is received by the probe 1 and converted into an electrical signal (photoacoustic signal). The probe body 10 has a case which can store therein a cable, an electronic circuit, a receiving element, etc. A processing circuit for amplifying or digitally converting the electrical signal may be provided in the probe case or the operation unit. When the photoacoustic probe is used in a hand-held manner, a grip part to be held by the hand is formed at the case. In this case, a receiving portion including the receiving element is usually provided on the opposite side to the grip part. The receiving portion is adapted to receive a photoacoustic wave generated from the object in response to irradiation of light emitted from the light source on the object.

The receiving portion may have a flat receiving surface or a projecting convex shape depending on the number of receiving elements. An arbitrary material may be used for the case, and for example a resin material may be used.

Any receiving element may be used if a photoacoustic wave can be converted into a photoacoustic signal, and for example a piezoelectric element or a CMUT may preferably be used. Ultrasonic wave is transmitted to/received by the object using the receiving element, so that ultrasonic echo measurement may be carried out in addition to photoacoustic measurement. The probe body has a contact portion 13 which contacts the object in addition to the receiving portion. The contact portion 13 protects the elements of the receiving portion and acoustically matches the receiving portion and the object. Photoacoustic waves which may cause noise with respect to light from the light source should preferably be avoided as much as possible. The contact portion 13 may be a resin, silicone, or rubber which can provide an acoustic matching function and an element protecting function, an acoustic lens which converges an acoustic wave, and a combination thereof.

An electrical signal output from the receiving element is processed by the operation unit 80 into information such as an initial sound pressure distribution and a light energy absorption coefficient distribution and formed into an image. The image is presented to the operator at a display unit 90. The operation unit 80 may be an information processing device (such as a PC and a workstation) which includes an operation resource such as a processor and a memory and operates according to a program command. For the imaging, any arbitrary image reconstruction method such as phasing addition and Fourier transform may be applied. In addition to the image reconstruction processing, the operation unit 80 may also function as a setting unit which sets conditions for photoacoustic measurement, a timing control unit which controls timing for irradiating light or acquiring a photoacoustic signal, and an angle control unit which controls the angle of the light source. The display unit 90 may be any arbitrary display device such as a liquid crystal display and an organic EL display.

An attachment for attaching the light source which will be described is provided at a tip end of the probe body 10, in other words, on the side of the contact portion in contact with the object. The attachment includes an attachment unit 2 as a part to be fixed to the probe body and a light source unit 3 which stores the light source. The attachment unit 2 includes an attachment part 21 and an attachment part 22. The attachment part 21 and the attachment part 22 are configured so that the probe body 10 can be held therebetween. The attachment part 21 and the attachment part 22 can be fixed so that these parts are not displaced with respect to the probe body 10 while having the probe body 10 held therebetween. The attachment unit corresponds to a probe attaching portion, and the light source unit corresponds to a light source attaching portion according to the present invention.

The light source unit 3 is attached to the attachment unit 2. The reference numeral 31 designates a light source container 31 for storing the light source in a detachable manner. An openable/closable cover 32 is provided on the light source container 31. The cover 32 has a sealing part (not shown) which keeps the inside of the light source container 31 airtight in a closed state. This prevents a matching agent used to acoustically match between the probe 1 and the object 100 from coming into the light source unit 3.

While the probe body and the light source are attached to the attachment, the receiving portion and the contact portion of the probe and the irradiating portion of the light source are arranged on the same side as the attachment as viewed from the object side. In a case where this state is viewed from the side of the contact part of the probe, the light source is attachable to and detachable from the attachment from the side different from the object (the side different from the object side where the contact portion contacts the object). In the illustrated photoacoustic probe, a part of the attachment parts 21 and 22, the receiving portion of the probe body 10, and a part of the light source container 31 including a window 34 are arranged substantially flush with one another. The receiving portion transmits/receives an acoustic wave from between the attachment parts 21 and 22. The light source generates light to be irradiated upon the object and emits the light through the window 34. In the illustrated example, the contact portion of the probe body held between the attachment parts is in direct contact with the object. However, the attachment may be provided with an acoustic wave transmitting film, so that the probe body may be prevented from being wet with a gel or water. The case in which the contact part is opposed to the object through the film is also included in the definition of the “contact between the object and the contact portion” herein.

FIG. 1B shows an attachment unit having a probe and a light source mounted thereto as viewed from the side in contact with the object. The contact portion 13 of the probe 1 is visible from the object side. The irradiating portion of the light source and the object are opposed through the window 34. An opening for transmitting light from the irradiating portion of the light source through the object is provided on the side of the light source unit 3 in contact with the object 100. The opening is provided with a transparent window 34 (window member) so that a matching agent for the object and the probe does not come into the light source unit 3. The window 34 may be made of acrylic which transmits light. The matching agent may be a gel, water, or an oil.

The light source container 31 is provided rotatably with respect to the attachment part 22 around a shaft 31a, as a rotation axis, which passes through the attachment part 22 and the light source container 31. The attachment part 22 has a slit 22a through which a rotation stopper 33 is attached to the light source container 31. The rotation stopper 33 and the light source container 31 are screw-fitted, and the attachment unit 2 and the light source unit 3 can be fixed or unfixed by turning the rotation stopper.

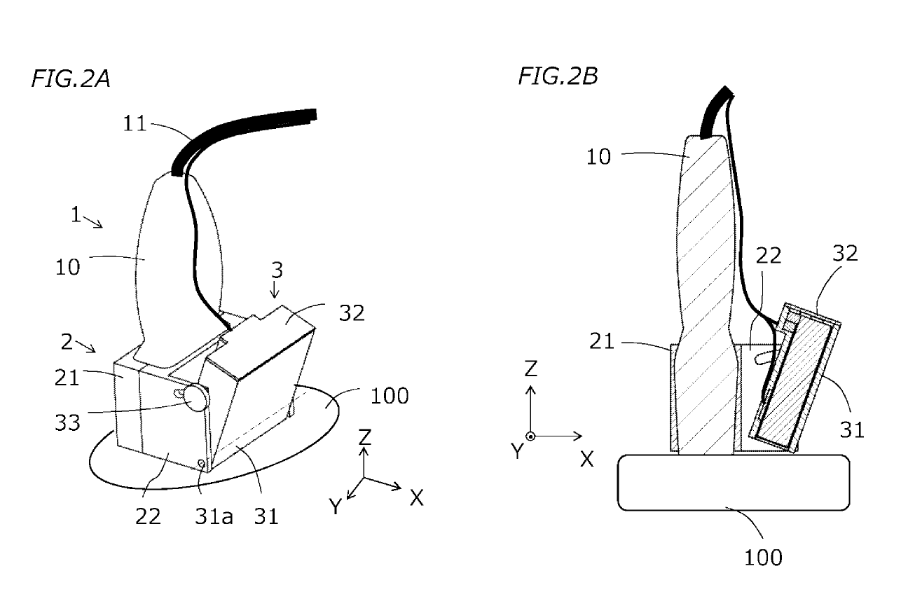

FIG. 2A is a perspective view showing a state in which the light source unit 3 is turned with respect to the attachment unit 2. FIG. 2B is a sectional view of the state shown in FIG. 2A. The light source unit 3 can be fixed at an arbitrary angle, and the position for irradiating light on the object 100 can be changed. The shaft 31a, the slit 22a, and the rotation stopper 33 constitute an angle change mechanism for changing an angle formed by the light source unit with respect to the attachment unit. Here, the combination of the slit and the rotation stopper is used as a stopper for fixing the angle but any of other combinations may be used.

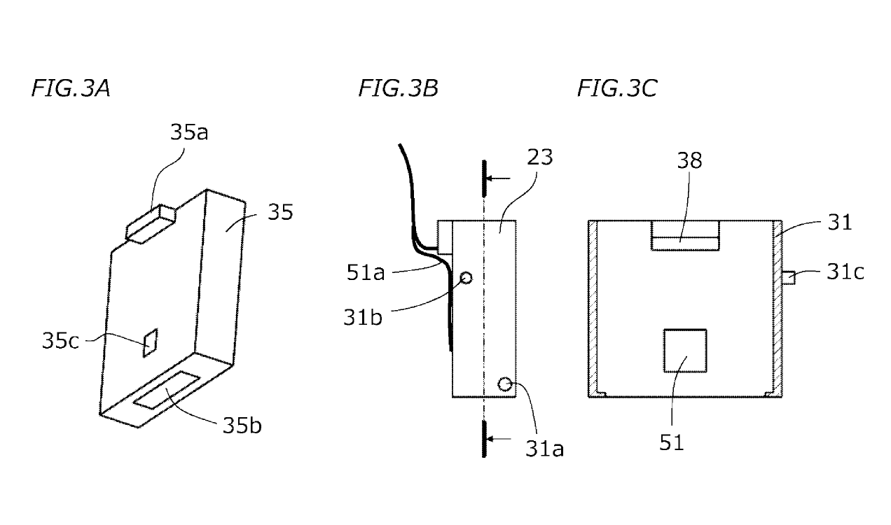

FIG. 3A is a perspective view of the light source 35 according to the embodiment. FIGS. 3B and 3C are a side view and a sectional view of the light source container 31. The light source 35 has a waveform which can easily be absorbed by the object. In a case where the object is a living body, the wavelength of light is preferably about in the range from 700 nm to 1200 nm which belongs to the near-infrared region called a biological window. The light in the region can reach relatively further into a deep part of the living body and allows information on the deep part to be obtained. A semiconductor laser (LD) or an LED is optimally used for the light source 35 in consideration of size reduction, but any of other light sources may be used.

In FIG. 3A, the reference numeral 35a designates a light source side connector which is connected to a container side connector provided at the light source container 31 to supply the light source 35 with power when the connector 35a is stored in the light source container 31. Through this connection, in addition to the supply of power, a signal cable for transmitting information on the light source 35 to the operation unit 80 is also connected. The information on the light source 35 may include a wavelength, the life of the light source, an amount of light, a light emitting frequency, and light emitting time per pulse. These kinds of information are received and displayed at the display unit 90, so that the state of the light source 35 can be checked and erroneous operation like inserting a light source with an unintended wavelength and irradiating light therefrom can be prevented. An acquired image and information on the light source 35 may be processed and stored in association with each other by the operation unit 80.

While the light source 35 is not inserted, the display unit 90 may indicate that the light source 35 is not inserted in addition to the information on the light source 35. In order to prevent insertion of an unintended light source 35 among a plurality of light sources, the light sources 35 may be partly painted in different colors for different wavelength, may be labeled with numbers or character strings representing wavelengths, or may be provided with irregularities.

Light from the light source 35 is emitted from an emission hole 35b. The light emitted to the emission hole 35b partly branches inside the light source 35 and is guided to a light amount measuring emission hole 35c provided at a side surface of the light source 35 and emitted therefrom. As shown in FIG. 3C, at the inner surface of the light source container 31, a light receiving portion 51 which receives the emitted light is provided to oppose the light amount measuring emission hole 35c in a case where the light source 35 is stored. The light receiving portion 51 and the operation unit 80 are connected by a light amount detecting cable 51a and information on the amount of light from the light source 35 detected at the light receiving portion 51 is transmitted to the operation unit 80. As the amount of light drops, the amount of light reaching the inside of the object decreases, which makes it difficult to acquire a signal from a deep position of the object and degrades the quality and reliability of images. However, in a case where a drop in the amount of light from the light source 35 is determined by carrying out the light amount detection, a current value for driving the light source 35 may be changed or the light source may be replaced with a new light source, so that a signal in the state with a lowered amount of light can be prevented from being acquired. The emission hole 35b corresponds to the irradiating portion, and the light amount measuring emission hole 35c corresponds to the second irradiating portion according to the present invention.

Note that it is not necessary to always direct light to branch toward the light amount measuring emission hole 35c. When at least object information is acquired, whether to branch or not may be switched so that the total amount of light can be emitted from the emission hole 35b. In this manner, the amount of light irradiated on the object in acquiring information on the object does not decrease, and the object information with a high SN ratio can be acquired. The location for detecting the amount of light is not limited to that according to the embodiment.

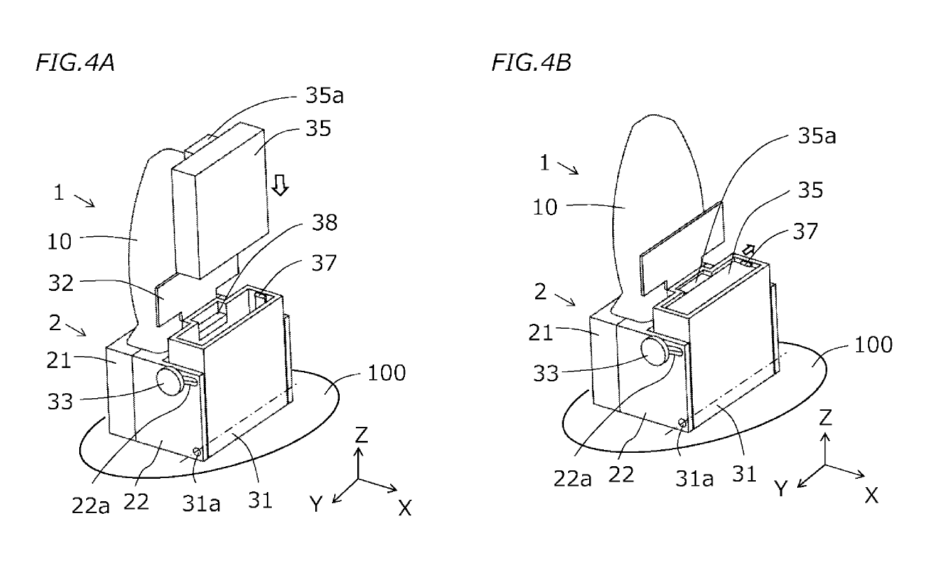

FIGS. 4A and 4B are schematic views showing how the light source 35 is inserted into the light source container 31. FIG. 4A is a view showing how the light source 35 is inserted after opening the cover 32. The light source container 31 includes therein a container side connector 38 connected to the light source side connector 35a of the light source 35. The cover 32 is opened and the light source 35 is inserted until the light source 35 is hooked by a claw 37 provided to keep the light source 35 from coming off.

FIG. 4B shows the state in which the light source 35 is inserted to be hooked by the claw 37. When the light source is inserted to be hooked by the claw 37, the light source side connector 35a and the container side connector 38 are connected. In this way, power is supplied to the light source 35 stored in a desired location of the light source container 31. Therefore, light is not irradiated from the light source 35 unless the light source 35 is incorporated in the light source container 31, which improves safety.

Then, a stored state in FIG. 1A is attained by closing the cover 32. In a case where the light source 35 is in the stored state, the light source 35 is constantly energized in a direction away from the light source container 31 by an energizing spring (not shown). In this way, by opening the cover 32 and unlocking the claw 37, the light source 35 is raised outside from the light source container 31, which makes it easier to detach the light source 35.

For at least a part of the area of the probe 1 or the attachment unit 2 in contact with the object 100, a material which increases abrasive resistance with the object 100 may be used, irregularities may be formed, or an adsorbing structure may be provided. This can prevent the probe 1 from being shifted from the object 100 during the light source 35 is replaced.

In this way, the photoacoustic probe 1 according to the embodiment allows the probe body and the light source to be attached to the attachment from the same side with respect to the object. In this way, the light source 35 can be replaced with a light source for irradiating light with a different wavelength while the probe 1 is kept in contact with the object 100. Therefore, since the same site of the object 100 can be photographed using a plurality of kinds of light having different wavelengths from one another, the reproducibility of the positional relation between the probe 1 and the object 100 may be improved. As a result, the measurement accuracy for oxygen saturation and the concentrations of substances may be improved.

As shown in FIGS. 2A and 2B, the light irradiation area on the object 100 can be changed by turning the light source unit 3. As the irradiation area can be changed, a reduced amount of light is irradiated on a photo-absorption spot (such as a mole) in vicinity of a desired photographing area which should be prevented from generating a signal. As a result, a signal with a reduced noise can be acquired. In the photoacoustic probe according to the embodiment, in the state in which the light source unit 3 is turned, the light source 35 can be replaced while the probe 1 is kept in contact with the object 100 by the function of the rotation stopper 33. Note that an angle detecting sensor such as an encoder may be provided, and rotation angle information on the light source unit 3 may be transmitted to the operation unit 80. The photoacoustic signal and the angle information are associated with each other, so that the light irradiation area for the photographed image can be determined. The estimation accuracy for a light amount distribution in calculating an absorption coefficient is improved.

First Modification

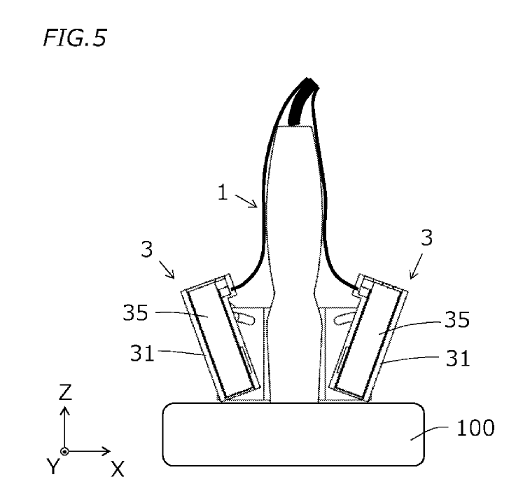

FIG. 5 is a sectional view of an exemplary arrangement having a light source unit 3 on either side of the probe 1. In this way, a plurality of light source units 3 are provided for a photoacoustic probe, the amount of light can be increased, which improves the SN ratio or the selection area for the light irradiation area can be increased. For example, light may be irradiated equally from both sides of the probe 1 or the rotation angles of the light source units 3 may be changed, so that light can concentrate at a desired irradiation location. As a result, a signal can be acquired from a deeper position or a wider range.

As another effect, the light sources 35 having different wavelengths from one another may be inserted in the light source units 3, and light can be irradiated alternately. In this way, photoacoustic signals derived from kinds of light having different wavelengths can be acquired without moving the probe 1 from the object 100.

Second Modification

In the above description, the light source unit 3 is turned around the shaft and the irradiation angle is changed. However, the direction in which the light source unit 3 is moved is not limited to the above. For example, there may be a moving mechanism for shifting the light source unit 3 in an acoustic wave transmitting/receiving direction by the probe 1 (in a substantially normal direction with respect to the surface of the object 100 in which the probe moves near or apart from the object). In this way, the light irradiation area can be expanded or contracted, and the amount of light per unit area can be adjusted accordingly.

The light source unit 3 may be shifted in a direction along the surface of the object. In this way, the irradiation area at the surface of the object can be changed.

In a case where the light source unit 3 is moved in a substantially normal direction with respect to the surface of the object or in a direction along the surface of the object, an arbitrary moving mechanism using for example a motor or a guide may be used to achieve the kinds of movements.

Third Modification

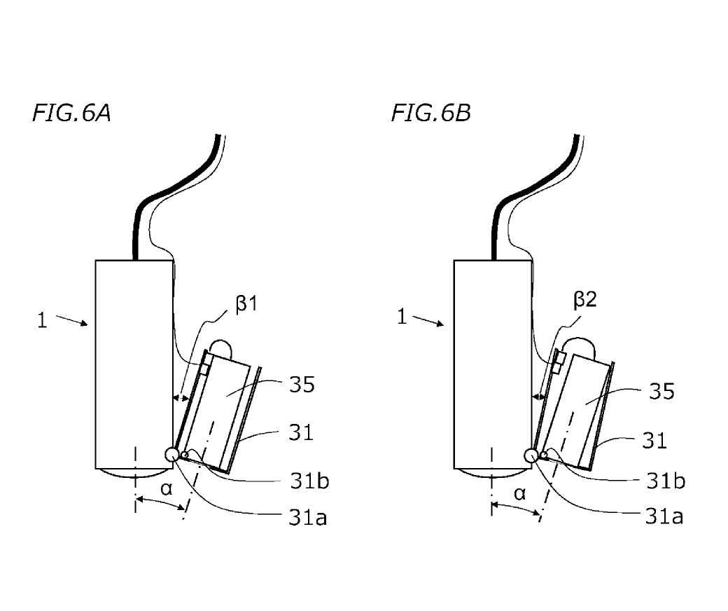

FIGS. 6A and 6B are schematic views of a photoacoustic probe further having a mechanism (a second angle change mechanism) for adjusting the angle of the light source 35 so that light from the light source 35 is directed in a fixed direction in a case where the angle of the light source container 31 is changed.

In FIG. 6A, the light source container 31 has an angle β1 with respect to the probe 1 around the shaft 31a as a rotation center. An encoder (angle detector) which is not shown may constantly detect the angle β1 of the light source container 31. The light source 35 can have its angle adjusted around a rotation shaft 31b further in the light source container 31. The angle adjustment based on the rotation shaft 31b of the light source 35 is automatically carried out by an angle controller which is not shown depending on the angle of the light source container 31. Here, the adjustment is controlled so that the angle of light irradiation upon the object 100 is a.

FIG. 6B shows a state in which the angle of the light source container 31 is changed to β2. The angle controller detects the change to the angle β2 on the basis of angle information output by the encoder and adjusts the angle of the light source 35 around the rotation shaft 31b. As a result, the light irradiation angle with respect to the object 100 is kept at α. The angle adjustment around the rotation shaft 31a or 31b can be carried out using an adjusting mechanism such as a motor.

Fourth Modification

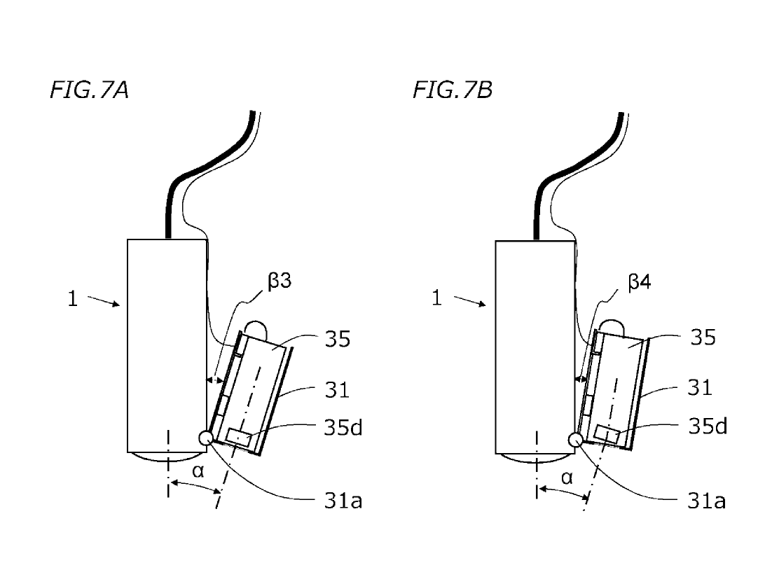

FIGS. 7A and 7B show a mechanism for adjusting the light irradiation angle stored in the light source 35. The light irradiation angle of the light source 35 is adjusted by an optical system 35d provided at a tip end of the irradiating portion. In FIG. 7A, the light irradiation angle is α when the angle of the light source container 31 is β3.

In FIG. 7B, the angle of the light source container 31 is β4. However, under the control of the optical system 35d, the light irradiation angle with respect to the object 100 becomes α, and therefore the light irradiation angle is kept at α. Here, the optical system 35d is provided in the light source 35, but the system may be provided outside the light emission end of the light source 35. The optical system 35d may be any system if the system can control the direction of light, and for example a movable mirror, a movable optical fiber, or a combination thereof may be used.

In this manner, in a case where the angle of the light source container 31 is changed by detaching the light source 35, light can be irradiated in a determined angle. As a result, a constant distribution of light irradiated on the object 100 can be obtained, which improves the reproducibility of an acquired signal. According to the modification, the angle can be adjusted as an angle controller (not shown) operates the optical system in response to an output from an encoder.

### Second Embodiment

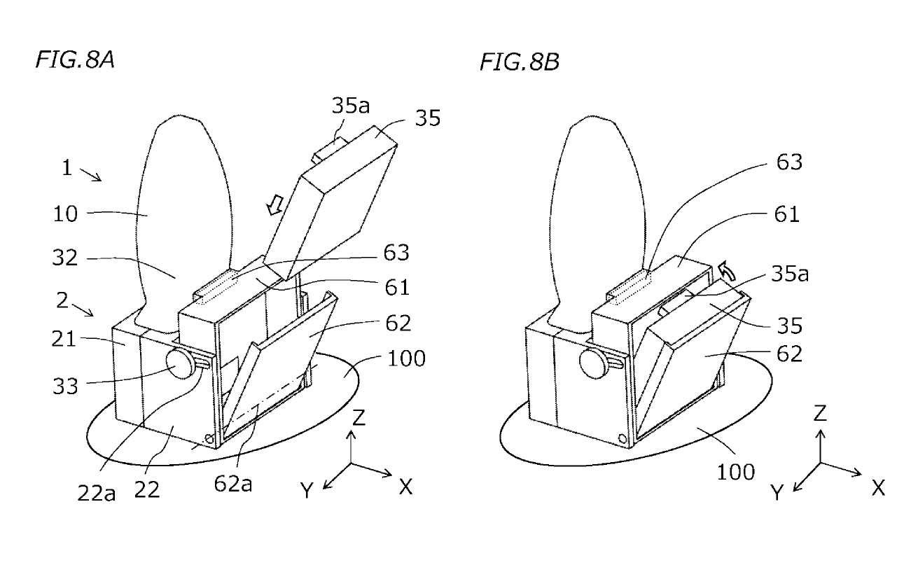

The structure according to the embodiment is shown in FIGS. 8A and 8B. A light source container according to the embodiment includes a light source container body 61 and a light source insertion guide 62. The light source insertion guide 62 rotates with respect to the light source container body 61 around a shaft 62a indicated by the chain-dotted line (FIG. 8A). In a case where the light source 35 is inserted, the light source 35 is inserted from above and set in the light source insertion guide 62. Then, the light source insertion guide 62 is turned and closed until the guide abuts against the light source container body 61 (FIG. 8B). By turning and closing the light source insertion guide 62 similarly to the structure shown in FIGS. 4A and 4B, the light source side connector 35a of the light source 35 and a container side connector 63 are connected and power can be supplied to the light source 35.

In the structure according to the embodiment, the light source 35 can be replaced or detached/attached without moving the probe 1 apart from the object 100. Therefore, the reproducibility of the positional relation between the probe 1 and the object 100 after the replacement of the light source 35 can be improved.

### Third Embodiment

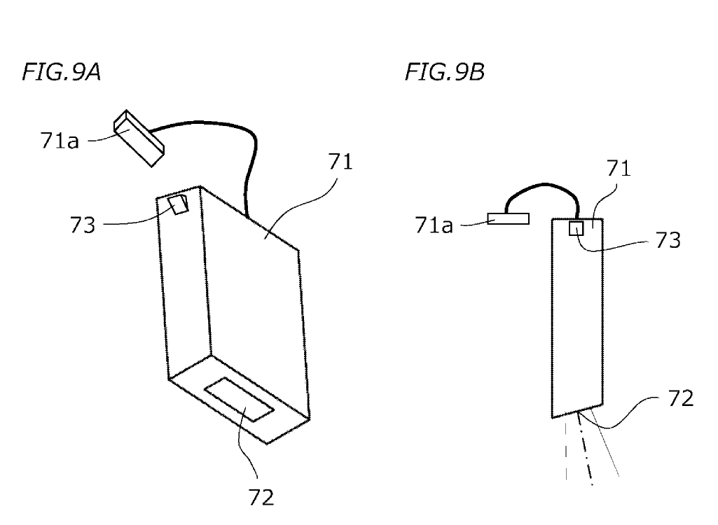

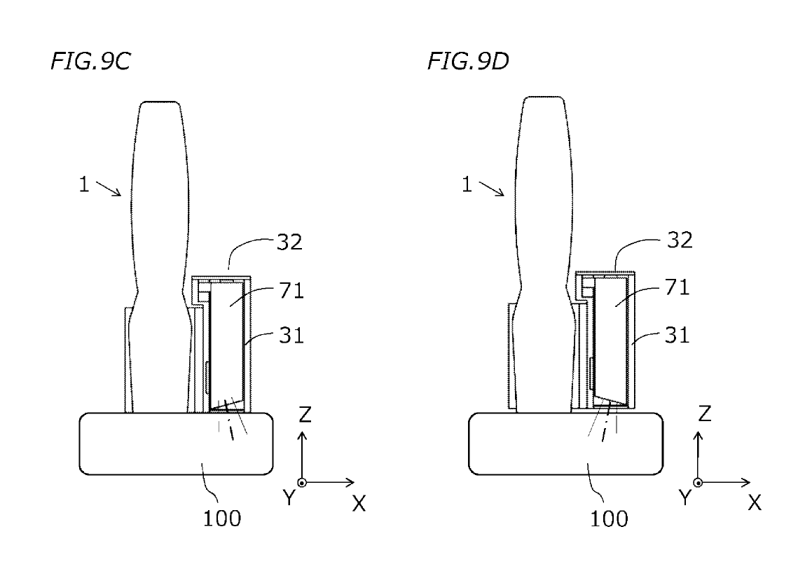

In the following description of the embodiment, the direction of light irradiation from a light source is inclined with respect to the longitudinal direction of the light source or the direction in which the light source is set. FIG. 9A is a perspective view of a light source 71 having an inclined light irradiation direction, and FIG. 9B is a side view thereof. The light emission hole 72 of the light source 71 is formed at a surface inclined with respect to the acoustic wave receiving surface or the surface of an object. The optical axis of the irradiation light has an angle as indicated by the chain-dotted line in FIG. 9B. More specifically, the irradiation light according to the embodiment enters obliquely with respect to the acoustic wave receiving surface or the surface of the object and is inclined with respect to the direction in which an acoustic wave is transmitted/received.

FIGS. 9C and 9D show the states in which the light source 71 is inserted in the light source container 31 in different directions. More specifically, in a case where the direction (up-down direction along the Z-axis) in which the light source 71 is attached to or detached from the light source container is a first direction, the light source 71 is inserted after being turned 180° around an axis provided in the first direction. The light emission direction may be changed between before and after the rotation of the light source 71 by inverting the insertion direction. A cable is drawn from the light source 71 and a connector 71a is provided at the tip end of the cable so that the light source 71 can be connected to the connector of the light source container 31 regardless of whether the light source 71 is inserted in the first direction or in the inverted direction.

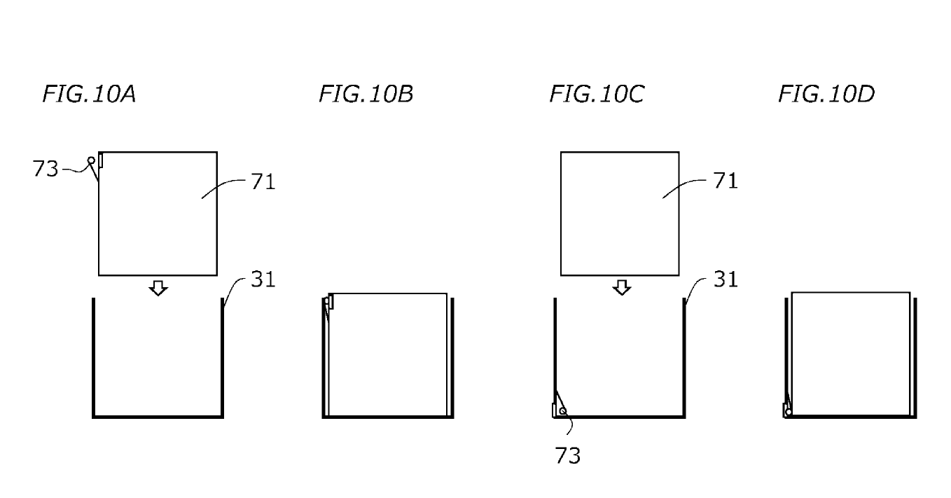

A limit switch 73 is provided at a side surface of the light source 71 in order to detect insertion of the light source 71 into a correct position in the light source container 31. FIGS. 10A and 10B are views showing an image of how the limit switch 73 is turned on/off by inserting the light source 71 into the light source container 31. According to the embodiment, light can be irradiated in case where connector 71a is connected even if the light source 71 is not inserted to a predetermined position. Therefore, the limit switch 73 is provided to detect insertion of the light source 71 in a correct position as shown in FIG. 10B, and then light can be emitted in response to the detected state, so that safety about light irradiation is secured.

The limit switch 73 may be provided in the light source container 31 as shown in FIG. 10C. In this case, light is also allowed to be emitted in response to detection of the light source inserted in a correct position as shown in FIG. 10D. The limit switch 73 can also be applied to the first and second embodiments in addition to this embodiment. Light irradiation may be controlled by detecting the opening/closing of the cover 32 of the light source container 31.

Note that according to the embodiments, a light source itself such as an LD and an LED is stored in the light source container 31. However, the present invention may be applied to a light source having a component other than an LD or an LED.

As in the foregoing, in a photoacoustic probe according to the present invention, a light source can be replaced while the probe is in contact with an object. As a result, the reproducibility of the positional relation between the probe and the object between before and after replacement of the light source can be improved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-229009, filed on Nov. 29, 2017, which is hereby incorporated by reference herein in its entirety.

What is claimed is:

1. A photoacoustic probe, comprising:
a light source which generates light to be irradiated on an object;
a probe body including a receiving portion which receives a photoacoustic wave generated from the object in response to irradiation of the light emitted from the light source on the object and a contact portion which contacts the object; and
an attachment to which the probe body and the light source are attached,
wherein the light source is attachable to and detachable from the attachment from a side different from the object side where the contact portion contacts the object.

2. The photoacoustic probe according to claim 1, wherein the attachment includes a probe attaching portion to which the probe body is attached and a light source attaching portion to which the light source is attached.

3. The photoacoustic probe according to claim 2, wherein the attachment has an angle change mechanism which changes an angle formed by the light source attaching portion with respect to the probe attaching portion.

4. The photoacoustic probe according to claim 3, wherein
the light source further includes an irradiating portion which irradiates the object with light, and
the light source attaching portion includes an angle detecting portion which detects a change in the angle formed by the light source attaching portion and a second angle change mechanism which further changes, on the basis of an output from the angle detecting portion, an angle at which the irradiating portion irradiates the object with light.

5. The photoacoustic probe according to claim 3, wherein the light source includes an optical system which changes a direction for irradiating the light.

6. The photoacoustic probe according to claim 3, wherein the angle change mechanism includes a shaft which passes through the probe attaching portion and the light source attaching portion and a stopper which fixes the angle formed by the light source attaching portion.

7. The photoacoustic probe according to claim 1, wherein in a case where a direction in which the light source is attached to or detached from the attachment is a first direction, the light source is inverted around an axis in the first direction and then attached to the attachment, so that a direction in which the light is irradiated changes.

8. The photoacoustic probe according to claim 1, wherein
the light source includes a second irradiating portion which emits light branched at the light source, and
the attachment includes a light receiving portion which can receive light from the second irradiating portion while the light source is attached.

9. The photoacoustic probe according to claim 2, wherein
the light source further includes an irradiating portion which irradiates the object with light,
the light source attaching portion is a light source container into which the light source is inserted, and
a part of the light source container opposed to the irradiating portion is provided with a window member which transmits the light.

10. The photoacoustic probe according to claim 1, wherein the light source is provided with a limit switch which detects an attachment state of the light source with respect to the attachment.

11. The photoacoustic probe according to claim 1, wherein the attachment is provided with a limit switch which detects an attachment state of the light source with respect to the attachment.

12. The photoacoustic probe according to claim 1, wherein a plurality of the light sources can be attached to the attachment.

13. The photoacoustic probe according to claim 1, wherein the attachment has a moving mechanism which moves the light source in a substantially normal direction to a surface of the object or in a direction along the surface of the object.

14. A photoacoustic probe, comprising:
a light source which generates light to be irradiated on an object;
a probe body including a receiving portion which receives a photoacoustic wave generated from the object in response to irradiation of the light emitted from the light source on the object and a contact portion which contacts the object; and
an attachment to which the probe body and the light source are attached,
wherein the light source is attachable to and detachable from the attachment while the contact portion is in contact with the object.

15. An attachment to which a light source and a probe body are attached, the light source generating light to be irradiated on an object, the probe body including a receiving portion which receives a photoacoustic wave generated from the object in response to irradiation of the light emitted from the light source on the object and a contact portion which contacts the object,
wherein the light source is attachable to and detachable from the attachment from a side different from the object side where the contact portion contacts the object.

16. An attachment to which a light source and a probe body are attached, the light source generating light to be irradiated on an object, the probe body including a receiving portion which receives a photoacoustic wave generated from the object in response to irradiation of the light emitted from the light source on the object and a contact portion which contacts the object,
wherein the light source is attachable to and detachable from the attachment while the contact portion is in contact with the object.
